(19) United States
(12) Patent Application Publication
(10) Pub. No.: US 2010/0136644 A1
(43) Pub. Date: Jun. 3, 2010

(54) METHODS FOR IMPROVING THE CULTIVATION OF AQUATIC ORGANISMS

(75) Inventor: Andrew Peter Musson, Central Hawkesbay (NZ)

Correspondence Address: KNOBBE MARTENS OLSON & BEAR LLP, 2040 MAIN STREET, FOURTEENTH FLOOR, IRVINE, CA, 92614, US

(73) Assignee: IOGENYX PTY LTD, Park Orchards (AU)

(21) Appl. No.: 12/527,405

(22) PCT Filed: Feb. 15, 2008
(86) PCT No.: PCT/AU2008/000190

§ 371 Date: Jan. 28, 2010

(30) Foreign Application Priority Data
Feb. 16, 2007 (NZ) 553276

(51) Int. Cl. C12N 13/00; C12N 1/20; C12N 1/12

(52) U.S. Cl. 435/173.1; 435/252.1; 435/257.1

(57) ABSTRACT

The present invention relates to electrochemical methods for altering a biological characteristic of an aquatic organism such as an algae or bacterium. The invention includes alterations to biological characteristics such as growth rate, membrane permeability and buoyancy.

### FIELD OF THE INVENTION

The present invention relates to methods for improving the cultivation of aquatic organisms, including single-cell organisms. More particularly, the methods include the use of electrical fields to affect the growth and certain other biological characteristics of aquatic organisms.

### BACKGROUND OF THE INVENTION

In nature, a large diversity of flora and fauna inhabit salt and fresh-water environments including the oceans, lakes, rivers, ponds, dams, reservoirs and the like. Life forms supported by aquatic environments range from simple organisms including bacteria and algae up to complex organisms such as plants and animals.

Apart from nature, the growth of organisms in aqueous environments is utilised industrially in many contexts such as agriculture (rice, and hydroponics). Biotechnology is also an economically important field that relies on the growth of prokaryotic and eukaryotic cells in aqueous environments. Examples include fermentation in the beer, wine and dairy industries, biofermentation, the production of recombinant proteins, the production of micro-organisms for cleaning biofilms, and the cultivation of both aerobic and anaerobic cells and organisms in the remediation of wastewater.

The products of fermentation are diverse, covering many needs in society. For example, cultivated algae are used in a wide variety of applications, such as an energy source in biofuels, as an ingredient in foodstuffs and nutritional supplements, and as a fertiliser.

Fish and crustacean farming is becoming more common and is an expanding industry that is becoming more important as open sea stocks become depleted or polluted, and the provision of a suitable controlled environment for breeding and farming fish and crustaceans and the like is a difficult and expensive business. Where enhanced growth of organisms and cells useful for the breeding and farming of aquatic animals can be achieved there will be an economic advantage.

There are many factors that must be taken into account when seeking to improve or optimize an aqueous environment for the growth of a target organism. Any one of a living organism's many metabolic requirements may be a limiting factor preventing the establishment, survival or reproduction of a species in a particular habitat. Aquatic plants, for example, are commonly limited in their distribution and abundance by the availability of nutrients, such as phosphorus or nitrogen. An abundance of phosphorus is of no use to the plant in the absence of nitrogen, and vice versa. Furthermore, the nutrients must be present in a bioavailable form.

Sufficient levels of dissolved gases such as oxygen and carbon dioxide levels may also be important for growth. The presence of oxygen may determine the extent to which some organic compounds are decomposed or preserved, and chemical processes at the oxic-anoxic interface can strongly influence the cycling of limiting nutrients for autotrophic and heterotrophic production, including nitrogen, phosphorus, and iron.

Other parameters such as temperature, light, the concentration of various ionic species, the presence of competitor organisms, and the like may also need to be taken into account in establishing or improving the growth of a microorganism in an aqueous environment.

In addition to the growth of microorganisms, the process of manipulating and harvesting those organisms is also important to production. For example, it may be necessary to synchronise the growth cycles of all organisms in a population such that the entire population can be harvested in a single point in time. It may also be necessary to alter the permeability, or even lyse the membrane and/or wall of a cell or organism under production. For example, it may be required to allow the passage of a solute in the aquatic environment to enter the cell, or to release a product made by an organism into the aquatic environment.

It is an aspect of the present invention to alleviate or overcome a problem of the prior art to provide apparatus and methods capable of improving the growth or manipulation of organisms in an aquatic environment.

### SUMMARY OF THE INVENTION

In one aspect, the present invention provides a method for altering a biological characteristic of an organism in an aqueous solution, the method comprising the steps of: (a) electrically contacting the aqueous solution with a first electrode device; (b) electrically contacting the aqueous solution with a second electrode device in a non-physical manner, and (c) passing an electric current between the first and second electrode devices, so as to establish an electric field in the aqueous solution. The biological characteristic may be growth, viability, the ability to reproduce, the timing of the cell cycle, the ability to assimilate a nutrient, the integrity or permeability of a membrane or wall of a cell of the organism, buoyancy or motility. It has been discovered that the application of an electrical field to aquatic organisms may result in alterations to growth rate, sporulation, the integrity of a membrane or wall of a cell of the organism (which may lead to electroporation, or even lysis). The ability to force a cell to float or sink has advantages for the harvesting of aquatic organisms.

While it is contemplated that the present methods may be used in respect of any aquatic organism (be it unicellular or multicellular), the organism is a typically a bacterium or an algae.

In one embodiment of the method the first electrode device is cathodic and the second electrode device is anodic. The second electrode device may be in contact with the ground, and may be an earth rod remote from the aqueous solution. Alternatively, the second electrode device comprises at least part of a wall of a means for containing the aqueous solution.

The first electrode device may comprise a non conductive housing and an electrode therein, the housing providing a conduit for flow of the aqueous solution therethrough such that the aqueous solution contacts the electrode.

The first and/or second electrode may be made from stainless steel, and may be in the form of an electrode mesh, a rod or a plate immersed in the aqueous solution.

In one embodiment of the method, the electrical current is DC.

The first and/or second electrode may include: a non conductive housing; one or more electrodes arranged within the housing; an inlet and an outlet in the housing for passage of aqueous solution therethrough such that the aqueous solution contacts each electrode; and means for connection of each electrode to a power source.

The electrode device may further include means for receiving a flow of oxidant through the housing such as an opening for connection to a supply of oxidising gas.

Where the electrode device comprises a non conductive housing, the device comprises one or more tubes, typically made from a plastic material such as polyvinylchloride.

Furthermore, the electrode of the electrode device may be mounted within a respective tube. In one embodiment the electrode is substantially coaxially mounted within a respective tube,

In one embodiment of the method the electrode device comprises two or more tubes in fluid communication with each other such that the aqueous solution flows from the outlet of one tube into the inlet of an adjacent tube. The tube may have a diameter d and an open end comprising one of the inlet and outlet, wherein the tube open end extends beyond the electrode by a distance up to about 4d. In one embodiment, gthe distance is between about 0.5d and about 4d.

In a second aspect the present invention provides an organism produced according to a method as described herein.

### BRIEF DESCRIPTION OF THE FIGURES

FIG. 1 is a schematic cross section showing a first embodiment of the method and system of the invention.

FIG. 2 is a schematic cross section showing a second embodiment of the method and system of the invention.

FIG. 3 is a schematic cross section showing a third embodiment of the method and system of the invention.

FIG. 4 is a schematic view showing a cross-section of an embodiment of the cathodic device of the invention.

FIG. 5 shows an outline of a process by which algae reproduce using organic matter, oxygen and sunlight.

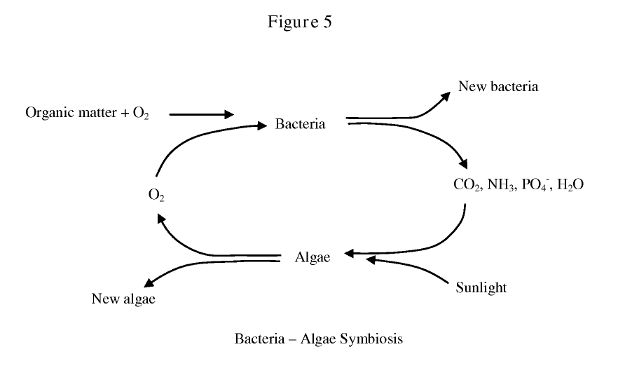

### DETAILED DESCRIPTION OF THE INVENTION

The present invention is predicated at least in part on the discovery that the establishment of an electrical field in an aquatic environment may be used to modulate the growth of organisms contained therein. Inventor has also discovered that electrical potentials may be useful in modulating the permeability of an organism in an aquatic environment, and also to regulate the cell growth cycle. Accordingly, in a first aspect the present invention provides a method for altering a biological characteristic of an organism in an aqueous solution, the method comprising the steps of: (a) electrically contacting the aqueous solution with a first electrode device; (b) electrically contacting the aqueous solution with a second electrode device in a non-physical manner, and (c) passing an electric current between the first and second electrode devices, so as to establish an electric field in the aqueous solution.

In the context of the present invention, the term “organism” includes any living cell or collection of cells, or any multicellular organism capable of living in an aqueous solution. The term includes any simple life form such as an alga, bacterium, yeast, fungus, mycoplasma, amoeba, or mammalian cell; or any more complex life form such as a fish, crustacean, or plant.

While discussion of the various aspects and embodiments of the invention is mainly directed to influencing and improving the cultivation of aquatic algae, it is to be understood that the invention is not limited to application to that specific type of organism.

The method is capable of altering a biological characteristic of the aquatic organism. As used herein, the term “biological characteristic” includes any one or more of the following characteristics:
(i) growth, including an alteration in any one or more of the following parameters: organism size, organism shape, organism surface area, or organism number, (ii) viability, (iii) the ability to reproduce, (iv) the timing cell cycle, (v) the ability to assimilate a nutrient, (vi) the integrity or permeability of a membrane or wall of a cell of the organism, including the ability of the organism to contain or exclude a solute or solvent, (vii) buoyancy, and (viii) motility.

As used herein, the term “altering” is intended to include any change in a biological characteristic that would not have resulted but for the application of the electric field in accordance with the present invention.

In the context of the present invention the term “aqueous solution” is intended to include any solution of any solute wherein water is the solvent, or at least the primary solvent. The aqueous solution may be naturally occurring, such as pond water, lake water, river water, creek water, stream water, ocean water, or sea water. The aqueous solution may be artificially created, such as a defined or undefined fermentation broth for the growth of bacteria, or a minimal essential medium for the growth of eukaryotic cells. It is not intended that the aqueous solution have a similar density or viscosity to pure water, and semisolid aqueous solutions (such as gels) are contemplated, for example.

The aqueous solution may be contained artificially by containment means such as a tank, pipeline, reservoir, dam, an incubation flask, a fermentation chamber, or a bioreactor; or by natural containment means such as that provided by a pond, lake, river, creek, stream, ocean, or sea. The means of containment may be a combination of natural and artificial means, such as a fish pen that is situated in a natural lake.

In one embodiment of the method the biological characteristic is growth of the organism. Inventor has demonstrated that the present methods are capable of improving the growth of algae in an aqueous environment. Accordingly the invention will find particular use in the economically important field of algaculture.

The majority of algae that are cultivated fall into the category of microalgae, also referred to as phytoplankton, microphytes, or planktonic algae. Macroalgae, commonly known as seaweed, also have many commercial and industrial uses, but due to their size and the specific requirements of the environment in which they need to grow, they do not lend themselves as readily to cultivation on a large scale as microalgae and are most often harvested wild from the ocean. However, it is nonetheless intended that the present methods are applicable to macroalgae.

When cultivating algae, several factors must be considered, and different algae have different requirements. The water must be in a temperature range that will support the specific algal species being grown. Nutrients must be controlled so algae will not be “starved” and so that nutrients will not be wasted. Light must not be too strong nor too weak.

In one embodiment, the method is carried out for cultivation of wild algae which can be cultured in raceway-type ponds and lakes. The present methods are particularly advantageous in such large-scale situations. The growing season is largely dependent on location and, aside from tropical areas, is typically limited to the warmer months. Improvements in growth as provided by the present methods may allow for a wider variety of environment conditions under which algae may be cultivated. A major benefit to this type of system are that it is one of the cheaper ones to construct, in the very least only a trench or pond needs to be dug. It can also have some of the largest production capacities relative to other systems of comparable size and cost. This type of culture can be viable when the particular algae in question requires (or is able to survive) some sort of extreme condition that other algae can not survive. For instance, Spirulina sp. can grow in water with a high concentration of sodium bicarbonate and Dunaliela salina will grow in extremely salty water. Open culture can also work if there is a simple inexpensive system of selecting out the desired algae for use and to inoculate new ponds with a high starting concentration of the desired algae. Some chain diatoms fall into this category as they can be filtered from a stream of water flowing through an outflow pipe. A “pillow case” of a fine mesh cloth is tied over the outflow pipe and most algae flow right through. The chain diatoms are held in the bag and used to feed shrimp larvae (in Eastern hatcheries) and to inoculate new tanks or ponds.

A variation on the basic “open-pond” system is to close it off, to cover a pond or pool with a greenhouse. While this usually results in a smaller system, for economic reasons, it does take care of many of the problems associated with an open system. It allows more species to be grown, it allows the species that are being grown to stay dominant, and it extends the growing season, only slightly if unheated, and if heated it can produce year round.

Algae can also be grown in a photobioreactor. A photobioreactor is a bioreactor which incorporates some type of light source. Virtually any translucent container could be called a photobioreactor, however the term is more commonly used to define a closed system, as opposed to an open tank or pond. Because these systems are closed, all essential nutrients must be introduced into the system to allow algae to grow and be cultivated. Essential nutrients include carbon dioxide, water, minerals and light. A pond covered with a greenhouse could be considered a photobioreactor. A photobioreactor can be operated in “batch mode” but it is also possible to introduce a continuous stream of sterilized water containing nutrients, air, and carbon dioxide. As the algae grows, excess culture overflows and is harvested. If sufficient care is not taken, continuous bioreactors often collapse very quickly, however once they are successfully started, they can continue operating for long periods. An advantage of this type of algae culture is that algae in the “log phase” is produced which is generally of higher nutrient content than old “senescent” algae. It can be shown that the maximum productivity for a bioreactor occurs when the “exchange rate” (time to exchange one volume of liquid) is equal to the “doubling time” (in mass or volume) of the algae.

While algae is often grown in monocultures using microbiological techniques to purify the desired strain, another approach has been used very successfully to produce algae feed for the cultivation of a variety of mollusks. Sea water is passed through filters to remove algae which are too large for the larvae being cultivated. Tanks in a green house, sometimes on a balcony in the mollusk house, are filled with the partially filtered water and nutrients are added. The tanks may be aerated and the water is used after only a day or two of growing. The resulting thin soup of mixed algae has been shown to be an excellent food source for larval mollusks. An advantage of this method of algaculture is the low maintenance requirements.

The practical advantage of increasing the growth rate of an algal culture is that more algal product is produced per unit time. Thus, a greater volume of product is achievable in a given volume of aqueous solution.

In one embodiment of the method the biological characteristic is the timing of the cell cycle. As will be understood by the person of skill in the art, the growth and reproduction of cells follows a predetermined cycle. For many cell types, the cell cycle consists of four distinct phases: G1 phase, S phase, G2 phase (collectively known as interphase) and M phase. M phase is itself composed of two tightly coupled processes: mitosis, in which the cell's chromosomes are divided between the two daughter cells, and cytokinesis, in which the cell's cytoplasm divides forming distinct cells. Activation of each phase is dependent on the proper progression and completion of the previous one. Cells that have temporarily or reversibly stopped dividing are the to have entered a state of quiescence called G0 phase.

The relatively brief M phase consists of nuclear division (mitosis) and cytoplasmic division (cytokinesis). In plants and algae, cytokinesis is accompanied by the formation of a new cell wall. The largest of all these processes is (interphase).

After M phase, the daughter cells each begin interphase of a new cycle. Although the various stages of interphase are not usually morphologically distinguishable, each phase of the cell cycle has a distinct set of specialized biochemical processes that prepare the cell for initiation of cell division.

The first phase within interphase, from the end of the previous M phase till the beginning of DNA synthesis is called G1 (G indicating gap or growth). During this phase the biosynthetic activities of the cell, which had been considerably slowed down during M phase, resume at a high rate. This phase is marked by synthesis of various enzymes that are required in S phase, mainly those needed for DNA replication. Duration of G1 is highly variable, even among different cells of the same species.

The ensuing S phase starts when DNA synthesis commences; when it is complete, all of the chromosomes have been replicated, i.e., each chromosome has two (sister) chromatids. Thus, during this phase, the amount of DNA in the cell has effectively doubled, though the ploidy of the cell remains the same. Rates of RNA transcription and protein synthesis are very low during this phase. An exception to this is histone production, most of which occurs during the S phase. The duration of S phase is relatively constant among cells of the same species.

The cell then enters the G2 phase, which lasts until the cell enters mitosis. Again, significant protein synthesis occurs during this phase, mainly involving the production of microtubules, which are required during the process of mitosis. Inhibition of protein synthesis during G2 phase prevents the cell from undergoing mitosis.

The term “post-mitotic” is sometimes used to refer to both quiescent and senescent cells. Nonproliferative cells in multicellular eukaryotes generally enter the quiescent G0 state from G1 and may remain quiescent for long periods of time, possibly indefinitely (as is often the case for neurons). This is very common for cells that are fully differentiated. Cellular senescence is a state that occurs in response to DNA damage or degradation that would make a cell's progeny nonviable; it is often a biochemical alternative to the self-destruction of such a damaged cell by apoptosis. Some cell types in mature organisms, such as parenchymal cells of the liver and kidney, enter the G0 phase semi-permanently and can only be induced to begin dividing again under very specific circumstances; other types, such as epithelial cells, continue to divide throughout an organism's life.

Thus, the methods of the present invention may act to synchronise cells at any one of any of the aforementioned stages or phases.

While the above passage is of general applicability, the present methods in some embodiments pertain to particular organisms. For example, the growth of bacteria involves the division of one bacterium into two identical daughter cells during a process called binary fission. Hence, local doubling of the bacterial population occurs. Both daughter cells from the division do not necessarily survive. However, if the number surviving exceeds unity on average, the bacterial population undergoes exponential growth. The measurement of an exponential bacterial growth curve in batch culture was traditionally a part of the training of all microbiologists; the basic means requires bacterial enumeration (cell counting) by direct and individual (microscopic, flow cytometry), direct and bulk (biomass), indirect and individual (colony counting), or indirect and bulk (most probable number, turbidity, nutrient uptake) methods.

In autecological studies, bacterial growth in batch culture can be modeled with four different phases: lag phase (A), exponential or log phase (B), stationary phase (C), and death phase (D).

During lag phase, bacteria adapt themselves to growth conditions. It is the period where the individual bacteria are maturing and not yet able to divide.

During the exponential phase (sometimes called the log phase), the number of new bacteria appearing per unit time is proportional to the present population. This gives rise to the classic exponential growth curve, in which the logarithm of the population density rises linearly with time. The actual rate of this growth depends upon the growth conditions, which affect the frequency of cell division events and the probability of both daughter cells surviving. Exponential growth cannot continue indefinitely, however, because the medium is soon depleted of nutrients and enriched with wastes.

During stationary phase, the growth rate slows as a result of nutrient depletion and accumulation of toxic products. This phase is reached as the bacteria begin to exhaust the resources that are available to them. At death phase bacteria run out of nutrients and die.

Thus, the methods of the present invention may act to synchronise bacterial cells at any one of any of the aforementioned stages or phases.

This basic batch culture growth model draws out and emphasizes aspects of bacterial growth which may differ from the growth of macrofauna. It emphasizes clonality, asexual binary division, the short development time relative to replication itself, the seemingly low death rate, the need to move from a dormant state to a reproductive state or to condition the media, and finally, the tendency of lab adapted strains to exhaust their nutrients.

Batch culture is the most common laboratory growth environment in which bacterial growth is studied, but it is only one of many. It is ideally spatially unstructured and temporally structured. The bacterial culture is incubated in a closed vessel with a single batch of medium. In some experimental regimes, some of the bacterial culture is periodically removed to a fresh sterile media is added. In the extreme case, this leads to the continual renewal of the nutrients. This is a chemostat also known as continuous culture. It is ideally spatially unstructured and temporally unstructured, in an equilibrium state defined by the nutrient supply rate and the reaction of the bacteria. In comparison to batch culture, bacteria are maintained in exponential growth phase and the grow growth rate of the bacteria is known. Related devices include turbidostats and auxostats.

Cell cycles can include events specific to certain genera or species of organism. For example, some species of alga include a sporulation step. This is the most common form of asexual reproduction in the algae. Sporulation refers to the process in which any cell of an organism produces one or more reproductive cells inside its cell walls. The original cell is termed a sporangium and the new cells are termed spores. Spores are often produced in large numbers for the rapid increase in population size. Inventor has demonstrated herein the ability of the present methods to synchronise the cell cycles of a population of algal cells, such that sporulation (or “ripening”) can be synchronised across the members of the population. The practical advantage of synchronising ripening is the ability to harvest the entire population of algae at their optimal productive stage.

In one embodiment of the method the biological characteristic is the integrity or permeability of a membrane or wall of a cell of the organism, including the ability of the organism to contain, exclude, admit or expel a solute or solvent.

The alteration of permeability may be useful for facilitating the entry of solutes into, or exit of solutes from, the cell of an aqueous organism by the process of electroporation (also known as electropermeabilization). This process is usually used in molecular biology as a way of introducing some substance into a cell, such as loading it with a molecular probe, a drug that can change the cell's function, or a piece of coding DNA. However, the method may be used on a larger scale, such as a bioreactor for the growth of bacteria.

Pores are formed when the voltage across a plasma membrane exceeds its dielectric strength. If the strength of the applied electrical field and/or duration of exposure to it are properly chosen, the pores formed by the electrical pulse reseal after a short period of time, during which extracellular compounds have a chance to enter into the cell. Excessive exposure of live cells to electrical fields can cause apoptosis and/or necrosis—the processes that result in cell death.

In molecular biology, the process of electroporation is often used for the transformation of bacteria, yeast, and plant protoplasts. In addition to the lipid membranes, bacteria also have cell walls which are different from the lipid membranes and are made of peptidoglycan and its derivatives. However, the walls are naturally porous and only act as stiff shells that protect bacteria from severe environmental impacts. If bacteria and plasmids are mixed together, the plasmids can be transferred into the cell after electroporation.

This procedure is also highly efficient for the introduction of foreign genes in tissue culture cells, especially mammalian cells. For example, it is used in the process of producing knockout mice, as well as in tumor treatment, gene therapy, and cell-based therapy. The process of introducing foreign DNAs into eukaryotic cells is known as transfection.

The biological characteristic of buoyancy may be related the integrity or permeability of a membrane or cell wall, given that these structures may act to regulate intracellular concentrations of salt, water, protein, fat, oil and gas. Alterations in buoyancy may be used practically to assist in the harvesting of aquatic organisms. For example, were buoyancy is increased, the organism will float to the surface of the aqueous solution, and may be conveniently scooped from the surface using a net, screen or similar contrivance. Conversely, where buoyancy is decreased the organism will sink to the bottom of the aqueous solution. The supernatant may then be siphoned away, leaving the concentrated organism for harvesting.

As discussed above, the present invention provides the ability to alter a biological characteristic of an organism. It will be appreciated that the invention is not limited to alterations that are necessarily positive to the overall health of the organism. For example, the present invention may still be useful where it is desired to inhibit the growth of an organism. Buoyancy of a photosynthetic organism may be decreased such that it is exposed to lower levels of light, thereby leading to a decreased growth rate. Where it is desired to completely kill an organism, the voltage may be applied such that the cell(s) of the organism are completely lysed, as described in the Examples herein.

The method comprises the use of first and second electrode devices. The first electrode device is in direct physical and electrical contact with the aqueous solution and is typically cathodic. It therefore typically exhibits a negative charge when in use. In one embodiment, the first electrode device comprises a non conductive housing and an electrode therein, with the housing providing a conduit for flow of aqueous solution therethrough such that the aqueous contacts the electrode. In another embodiment, the first electrode device may comprise an electrode mesh or plate immersed in the aqueous solution.

The second electrode device is in electrical, but not physical, contact with the aqueous solution and is typically anodic. It therefore typically exhibits a positive charge when in use. The second electrode device may be in contact with the ground and may comprise an earth rod remote from the aqueous solution. In another embodiment, the second electrode device may comprise at least part of a wall of containment means holding the aqueous solution.

The second electrode device is accordingly not in direct physical contact with the aqueous solution, although it is in electrical contact. This may be effected by burying the second electrode device in the ground away from the aqueous solution (for example, in the case of treating a large body of water outdoors, eg a lake) or the electrode may comprise an external wall of a containment means holding the aqueous solution (for example, where a smaller volume of aqueous solution is being treated, eg a tank or pond). In either case, in order for there to be electrical contact between the aqueous solution and the second electrode device, the containment means surrounding the aqueous solution (eg walls, surrounding ground etc) should be electrically conductive.

An important feature of the present invention is that by not physically contacting the aqueous solution with the second electrode device the chemistry of the aqueous solution can be controlled such that a biological characteristic of an organism in the aqueous solution can be altered. Without wishing to be limited by theory, it is believed that while half cell reactions associated with the first electrode device are able to proceed, those associated with the second electrode device are not because relevant aqueous species cannot reach the point of charge of the second electrode device and there is accordingly insufficient ion migration for half cell reactions associated with the second electrode device to proceed to completion. Instead, the second electrode and the region between the second electrode and the inner surface of the containment means becomes a half cell.

Another associated advantage of not having the second electrode device in direct physical contact with the aqueous solution is that galvanic corrosion of electrodes is minimised.

As stated, it is preferred that the first electrode device is cathodic and the second electrode device is anodic.

A cathodic first electrode device is preferred for a number of reasons. Firstly, by virtue of an anodic second electrode device not being in physical contact with the aqueous solution, there is insufficient ion migration for completion of half cell reactions ordinarily associated with anodes. In particular half cell reactions involving production and outgassing from solution of oxygen as a gaseous phase are typical anodic half cell reactions during electrolysis of water. However, in one embodiment of the method of the present invention, due to the anodic second electrode device being outside the water, anions involved in those half cell reactions are unable to reach the anodic point of charge. There is accordingly insufficient current density within the aqueous solution for reactions to result in release from solution (“gassing off”) of oxygen as a gaseous phase. Accordingly, oxygen is dissolved in solution, resulting in an oxygen enriched solution which may be supersaturated with oxygen. This environment is particularly advantageous in the treatment of organic impurities.

Another reason why a cathodic first electrode device is preferred is because most inorganic contaminants are cationic (especially metal ions) meaning that cations will migrate to the cathode and may undergo half cell reactions and/or precipitation as salts there which can remove them from solution.

Furthermore, the stability of common electrode materials is greater under cathodic, rather than anodic, conditions. Many common electrode metals would be susceptible to oxidation (galvanic corrosion) under anodic conditions, which would further contaminate the aqueous solution with hydroxides of the anode metal. Accordingly, where the first electrode device is anodic, it is preferably made from oxidation resistant material, such as platinum.

In some embodiments, the polarity of the electrodes may be reversed. For example, where the first electrode device is cathodic, and the second electrode device is anodic, the polarity of the electrodes may be reversed, at least temporarily, for the purpose of periodic cleaning of the electrodes to remove matter deposited thereon during electrolysis, for example metal salts.

When an electrical current is passed through the electrodes, an electric field is set up in the aqueous solution. The electrical current is typically DC, although an AC current may be used in some applications such as where polarity reversal is required.

The electrode device may comprise a non-conductive housing and one or more electrodes arranged within the housing. The housing may include one or more tubes. The tube(s) are typically made from a plastics material, and in one embodiment the tube(s) are made from polyvinylchloride. The housing may be non-perforated, having substantially solid walls, which may provide the advantage of minimising fouling of the electrode.

The electrode may a rod, or includes a rod, the rod being solid or hollow. Typically the rod is made from stainless steel. In one embodiment of the method, the electrode is arranged substantially coaxially within a respective tube.

The electrode device may also include an inlet and an outlet in the housing, for passage of organisms-containing aqueous solution therethrough such that the aqueous solution contacts each electrode, and means for connection of each electrode to a power source.

In one embodiment of the method the electrode is mounted within a respective tube having an open end which functions as either an inlet or outlet for the aqueous solution. The open end of the tube extends beyond the electrode by an amount sufficient to minimise ionic deposits on the electrode. Preferably, the first end of the electrode is adapted for connection to the power source and the open end of the respective tube extends beyond the second free end of the electrode.

The inventor has found that by distancing the free end of the electrode from the open end of the tube, the amount of fouling caused by ionic deposits on the electrode can be reduced, thereby minimising the likelihood of obstruction of flow past the electrode. The inventor has also found that optimum results are obtained when the open end of the tube is spaced from the electrode by an amount up to 4 times the diameter of the tube, and in some embodiments of the method from 0.5 to 4 times the diameter. Without wishing to be limited by theory, it is believed that by distancing the electrode from the open end of the tube, the current path lines within the tube become more focussed, resulting in a relatively concentrated electric field within the tube compared with that outside the tube. Ionic deposits then tend to form on the outside of the tube, rather than on the electrode.

A further advantageous effect of distancing the free electrode end from the open end of the tube is that the focussed electric field reduces power requirement significantly, such as by up to 70-80%. For example, where the tube has a diameter of 100 mm, and the free end of the electrode is spaced approximately 100 mm from the open end of the tube, the current requirements are reduced from approximately 250 mA to 50 mA.

The electrode device may further comprise means for receiving flow of a reactive fluid, such as an oxidant, through the housing. The means for receiving a flow of reactive fluid may comprise an opening for connection to a supply of gas, for example air or other oxidising gas, and may be adapted for connection to a supply of compressed air to form an airlift pump.

The introduction of an oxidant during the electrolytic method greatly enhances the solubility of the oxidant, such as oxygen in solution, particularly where the first electrode device is cathodic. The introduced oxygen, together with dissolved oxygen produced during electrolysis, can result in a solution supersaturated with oxygen. Production of reactive oxygen species, oxyanions and free radicals is favoured.

In one embodiment of the method, the electric field is of sufficient strength and/or duration to increase the supply of oxygen to the organism. Where the organism requires dissolved oxygen to grow, this embodiment will increase the growth or viability of the organism.

In another embodiment of the method, the electric field is of sufficient strength and/or duration to increase a membrane electrochemical potential of the organism.

In one embodiment of the method, the electric field is of sufficient strength and/or duration to co-localise the organism and a nutrient required by the organism in the aqueous solution. Bringing an organism into closer proximity with a nutrient (be it essential or inessential) increases the availability of that nutrient to the organism thereby assisting growth.

The skilled person is capable of varying parameters such as distance between electrodes, current, voltage and time in order to effect or optimise any desired result on a biological characteristic. In one embodiment of the method, the distance between electrodes is selected from the group consisting of at least about 1, 2, 3, 4, 5, 6, 7, 8, 9, 10, 15, 20, 25, 50, 75, 100, 250, 500, 750 and 1000 meters. In a further embodiment the voltage is selected from the group consisting of at least about 1, 2, 3, 4, 5, 6, 7, 8, 9, 10, 15, 20, 25, 50, 75, 100, 250, 500, 750 and 1000 volts. For increased growth of algae, a voltage of less than 24V is typical, while for membrane permeablization or lysis of a cell a voltage of great than about 24V is typically used. In yet a further embodiment of the method, the electric field is applied for a period selected from the group consisting of at least about 1, 2, 3, 4, 5, 6, 7, 8, 9, 10, 15, 20, 25, 50, 75, 100, 250, 500, 750 and 1000 minutes, or 1, 2, 3, 4, 5, 6, 7, 8, 9, 10, 11, 12, 13, 14, 15, 20, 25 and 30 days, or 1, 2, 3, 4, 5, 6, 7, 8, 9, 10, 11 and 12 months. It is emphasised that the electric field may or may not be applied continuously for any period, and may be switched on or off for periods of time. It is also emphasised that voltage (or indeed any other parameter) may be varied over the course of any duration.

It will be understood that a single desired outcome in terms of biological characteristic may be obtained by manipulation of any two or more of the aforementioned parameters. For example, a decrease in voltage may be compensated for by an increase in time, and vice-versa.

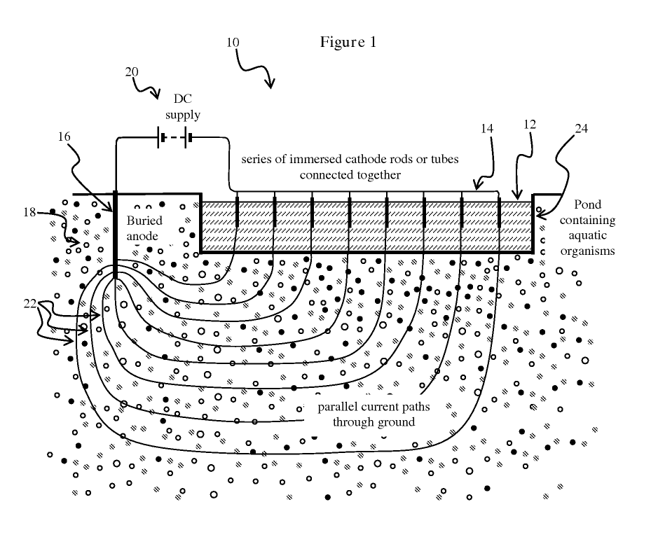

In one embodiment of the method, the cathodic device is placed within the aqueous solution. By placing the cathodic device within the body of aqueous solution there is a direct current path between the walls of the containment means and the cathodic device such that an electric field is produced which allows the migration of ions within the aqueous solution to their respective electrodes. Anion species will migrate towards the walls of the container and cation species will migrate towards the cathodic device. By direct and/or indirect electrolysis, one of the products of the anion migration may be oxygen, a necessary element for aerobic respiration. This may lead to increased aerobic bacterial activity, leading to increased nutrient availability for plant and algal organisms, leading to increased production of these organisms, according to FIG. 1, which shows the oxygen coming in at top left leading to new algae at bottom left.

In another embodiment of the method the cathodic surface area is less than about 70, 65, 60, 55, 50, 45, 40, 35 or 30% of the surface area of the inside wall of the containment means. In one embodiment, the cathodic surface area is less than about 65% of the surface area of the inside wall of the containment means. By keeping the cathodic surface areas small in relation to the area of the inside wall of the containment means, a non-uniform potential gradient may be established within the aqueous solution such that the potential gradient increases with proximity to the cathodic device. This is shown in the FIG. 1, bacteria in the region of the cathodic device may come under the influence of this increased potential gradient with the effect that the membrane electrochemical potential within the cell may be influenced leading to the stimulation of several fundamental physiological processes, including ATP synthesis, flagellar rotation, and growth yield. Thus the influence of the cathode may lead to increased bacterial size and activity, in turn leading to increased production of algae

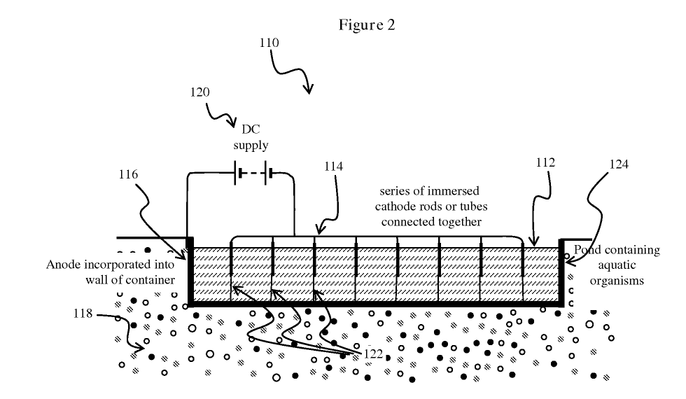

In one embodiment of the method, the electric field is non-uniform. By means of the non-uniform electric field lines, shown dotted in FIG. 2, it is seen that there is a greater electric field where the lines are closer together. This may have the effect of attracting nutrients and microscopic organisms to these regions of higher electric field, where increased biological activity will occur. This in turn may lead to increased production of preferred species.

Once the desired algal growth has been achieved, one or two further steps may be performed, both using the method of the invention:

Step 1. By switching off the power supply (DC in one embodiment) the algae are stimulated to enter a resting spore stage known as ripening. This process may take a number of days and is advantageous to the process of harvesting.

Step 2. By switching on the power supply with increased or reversed polarity voltage, which may consist of pulses of short duration and high energy or AC, the algae are lysed and will sink from suspension, enabling a scavenging process to facilitate harvesting.

When algae is killed in the resting spore stage, the oil can be harvested from the top of the aqueous solution because a proportion has been gathered up with the cell and easily jettisoned. Algae that are not in the resting spore stage will lyse and sink to the bottom retaining much of the lipid. They may be harvested from the bottom of the vessel so lipid can be retrieved by other processes or for any other use.

It will be understood that any treatment of organisms by the present methods may or may not take place in the containment means in which the organisms have been grown or maintained. For example, cells may be grown in a bioreactor under no electric field, and subsequently transferred to a separate vessel for subsequent lysis or electroporation by the application of an electric field as described herein.

While many types of electrodes (such as solid rod-type electrodes, meshes, plates etc) will find use in the context of the present methods, certain embodiments are disclosed infra. In the following discussion of the drawings, like reference numerals refer to like parts.

FIG. 1 shows a schematic cross section 10 of a first embodiment of the method and system of the invention, as used to influence and improve the cultivation and growth of aquatic organisms in an electrically conducting container 24 which may be a pond dug in open ground containing a body of water 12. A cathodic device comprising a series of rods or tubes electrically connected together 14 is immersed in and covers an area within the body of water 12. An anodic device comprising an anode rod 16, is buried in the ground 18 surrounding the body of water 12 The ground 18 effectively acts as a water containment means for the body of water 12. The cathodic device 14 and anode rod 16 are connected to the negative and positive terminals respectively, of a power source comprising a DC voltage supply 20. The DC voltage supply 20 can be adjusted to provide a voltage of between 0 and −100 volts, thereby establishing an electric field in the body of water 12 and surrounding ground 18, shown by current path lines 22. The voltage is adjusted until an electric field of sufficient strength and duration is achieved to effect one, some or all of the following processes:
(i) Increased supply of oxygen to supply the needs of aerobic respiration to beneficial aerobic species.
(ii) Increased cellular membrane electrochemical potential leading to increased fundamental physiological processes and growth yield.
(iii) Efficient localised concentrations of species and their nutrients.

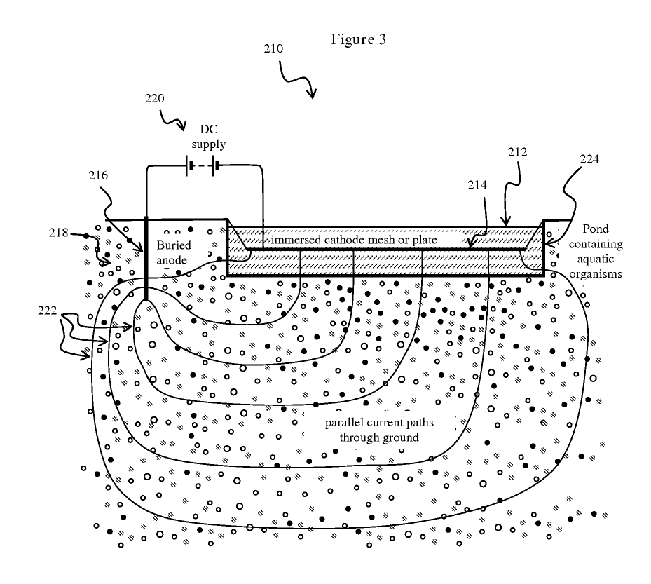

In the following description of FIGS. 2 and 3 illustrating the second and third embodiments respectively of the method and system of the invention, discussion will focus on those aspects of the embodiments which differ from those of the first embodiment.

In FIG. 2, the body of water 112 is provided within a water containment means comprising an electrically conductive container 124. The outer wall is not in physical contact with the water. Accordingly, the electric field, indicated by current path lines 122, is wholly within the conducting container 124 and the body of water 112 and does not extend out to the surrounding ground 118. Accordingly, the current passes from the power supply 120, through the anodic device 116 incorporated into the wall of the container 124, into the body of water 112, then to the immersed cathodic device comprising a series of rods or tubes electrically connected together 114 and back to the power supply 120.

FIG. 3 shows a variation in which the cathodic device 214 is a substantially planar immersed mesh or plate. Accordingly, the current path lines 222 pass from the power supply 220, through the buried anode 216 through the ground 218, through the wall of the electrically conductive container 224, into the body of water 212, then to the immersed substantially planar cathode 214 and back to the power supply 220.

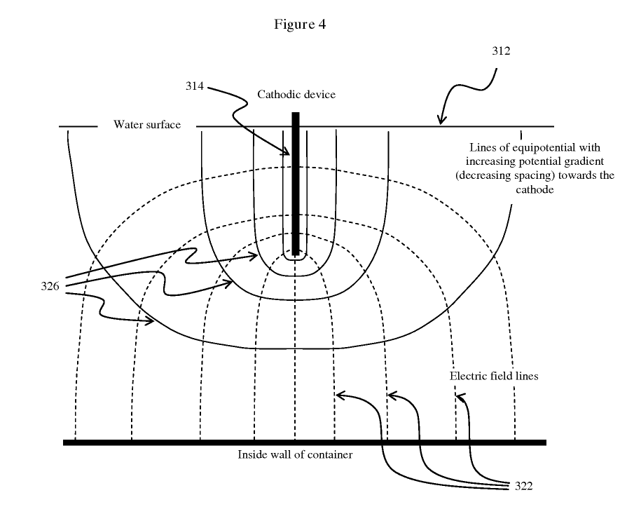

FIG. 4 shows a cross-section of one of the cathodic rods 314 immersed in the water 312 and showing the shapes of the electric field lines 322 and the equipotential surfaces 326. The field lines 322 are seen to be closer together in the region substantially below the cathodic rod, and the equipotential surfaces 326 are seen to be closer together in the region more proximal to the cathodic rod 314. These non-uniformities influence both the cellular membrane electrochemical potential and the localised concentrations of species and their nutrients.

The present invention will now be more fully described by reference to the following non-limiting examples.

### EXAMPLES

### Example 1

### Application of 12V Electric Field to Algae

50 litres of wastewater from an oxidation pond containing wild algae was subjected to the invention with 12V maintained at the electrodes over a six week period in a small test pond. 1 litre of wastewater from the same source was added daily. This volume replaced losses from evaporation.

Algal densities were maintained and increased slowly over the six week period. There was no sign of algae dying off and sinking.

At the end of the six week period the system was turned off and observed. After 3 days a uniform resting spore stage was achieved indicated by a change in colour to a golden yellow. This is the evidence for synchronisation in that resting spore stage can be triggered across the whole population.

The system was re-energised at 12V, resulting in the lysing of the cells, the sinking of biomass to the bottom of the pond, and the floating of lipids, indicated by observable oil on the surface.

### Example 2

### Application of 48V Electric Field to Algae

This trial was conducted as described in Example 1 with identical results through the six week period.

The test pond of dimensions 600 mm×400 mm×480 mm contained 50 litres of waste water. The electrified pond cathode was composed of stainless steel and suspended vertically in the water. The bottom of the rod was 10 mm from the floor of the pond. A stainless steel rod-type anode of 10 mm×700 mm was buried in the ground at a distance of 5 meters from the edge of the pond. Algae under cultivation were wild species naturally occurring in the waste water from an oxidation pond. Growing voltage was 12V, and the current 250 mA. The algae were lysed with 48V without turning off the system and before resting spore stage was initiated. This resulted in the sinking of the dead biomass which concentrated itself in the region of maximum electric field.

Lysis occurs through disruption of cell membranes. This was achieved at a relatively high voltage. At lower voltages, lysis did not occur, and we deduce that sublethal, transient and localised disruption would have occurred.

Finally, it is to be understood that various other modifications and/or alterations may be made without departing from the spirit of the present invention as outlined herein.

1. A method for altering a biological characteristic of an organism in an aqueous solution, the method comprising the steps of:
(a) electrically contacting the aqueous solution with a first electrode device;
(b) electrically contacting the aqueous solution with a second electrode device in a non-physical manner, and
(c) passing an electric current between the first and second electrode devices, so as to establish an electric field in the aqueous solution.

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method according to claim 1 wherein the biological characteristic is selected from the group consisting of growth, viability, the ability to reproduce, the timing of the cell cycle, the ability to assimilate a nutrient, the integrity or permeability of a membrane or wall of a cell of the organism, buoyancy and motility.

33. The method according to claim 32, wherein the growth is increased growth rate.

34. The method according to claim 32, wherein the timing of the cell cycle is the synchronisation of the cell cycle in a population of organisms, or the synchronisation of sporulation in a cell of an organism.

35. The method according to claim 32, wherein the alteration of the integrity of a membrane or wall of a cell of the organism lyses the cell, or allows for electroporation of the cell.

36. The method according to claim 1, wherein the organism is a bacterium or an algae.

37. The method according to claim 1, wherein the first electrode device is cathodic and the second electrode device is anodic.

38. The method according to claim 1, wherein the second electrode device is in contact with the ground.

39. The method according to claim 38, wherein the second electrode device comprises an earth rod remote from the aqueous solution.

40. The method according to claim 1 wherein the second electrode device comprises at least part of a wall of a container having the aqueous solution.

41. The method according to claim 1, wherein the first electrode device comprises a non conductive housing and an electrode therein, the housing providing a conduit for flow of the aqueous solution therethrough such that the aqueous solution contacts the electrode.

42. The method according to claim 1, wherein the first electrode device is an electrode mesh, a rod or a plate immersed in the aqueous solution.

43. The method according to claim 1, wherein the first or second electrode comprises: a non conductive housing; one or more electrodes arranged within the housing; an inlet and an outlet in the housing for passage of the aqueous solution therethrough such that the aqueous solution contacts each electrode; and a connector configured to attach each electrode to a power source.

44. The method according to claim 1, wherein the electrode device further comprises an opening for receiving a flow of oxidant through the housing.

45. The method according to claim 43 wherein the non conductive housing of the electrode device comprises one or more tubes made from a plastic material or polyvinylchloride.

46. The method according to claim 1, wherein each electrode of the electrode device is mounted within a respective tube.

47. The method according to claim 46, wherein the electrode is substantially coaxially mounted within a respective tube.

48. The method according to claim 1, wherein the electrode device comprises two or more tubes in fluid communication with each other such that the aqueous solution flows from the outlet of one tube into the inlet of an adjacent tube.

49. The method according to claim 48, wherein the tube has a diameter d and an open end comprising one of the inlet and outlet, wherein the tube open end extends beyond the electrode by a distance up to about 4d.

50. An organism produced according to a method according to claim 1.
